(19) United States
(12) Patent Application Publication
(10) Pub. No.: US 2016/0207023 A1
(43) Pub. Date: Jul. 21, 2016

(54) OPTICAL EXCITATION OF CHEMICAL SPECIES FOR ENHANCED CHEMICAL REACTION

(71) Applicant: Roger R. Dube, Pittsford, NY (US)

(72) Inventor: Roger R. Dube, Pittsford, NY (US)

(21) Appl. No.: 14/972,057

(22) Filed: Dec. 16, 2015

Related U.S. Application Data
(63) Continuation-in-part of application No. 14/302,328, filed on Jun. 11, 2014, now abandoned.
(60) Provisional application No. 61/833,764, filed on Jun. 11, 2013.

(51) Int. Cl. B01J 19/12; C07C 2/00

(52) U.S. Cl. CPC B01J 19/127; C07C 2/00; B01J 2219/0875

(57) ABSTRACT

An invention is provided for molecular excitation via monochromatic light at a particular wavelength. The invention includes providing a chemical to an illumination chamber, and illuminating the chemical with monochromatic light of a predefined wavelength. As a result, the chemical is placed in an excitation state that results in the molecules of the chemical being more likely to react with other molecules. Thereafter the chemical is provided to a reaction chamber, wherein the molecules of the chemical bond with other molecules in a predefined manner.

### CROSS REFERENCE TO RELATED APPLICATIONS

This application is a continuation-in-part of U.S. patent application having Ser. No. 14/302,328, filed on Jun. 11, 2014, by inventor Roger R. Dube, and entitled “Optical Excitation of Chemical Species For Enhanced Chemical Reaction,” which claims the benefit of U.S. Provisional Patent Application having Ser. No. 61/833,764, filed on Jun. 11, 2013, and entitled “Optical Excitation Of Chemical Species For Enhanced Chemical Reaction,” both of which are hereby incorporated by reference.

### BACKGROUND OF THE INVENTION

“Gas to Liquids” (GTL) is a term used to describe a variety of processes that convert an input gas (for example, methane) to a longer chain hydrocarbon (ideally, a liquid). The vast majority of GTL processes in use and under development today employ heat and physical catalysts. One such example is the Fischer-Tropsch (F-T) process, named after the two inventors of the process. The F-T process burns some of the source gas (natural gas, consisting primarily of methane) to heat up a chamber of natural gas, which is then allowed to come into contact with a physical catalyst. The rapid movements of the heated molecules increase the likelihood that they will encounter the catalyst. On the surface of the catalyst, the molecules have a lower activation potential—that is, the barrier to a reaction (such as the combining of two methane molecules) is reduced, and longer chain hydrocarbons are created at a much higher rate than would occur otherwise.

One drawback to this conventional approach to producing longer chain hydrocarbons is that the process is very inefficient. Large amounts of heat must be produced to achieve the throughputs desired. A second drawback of this approach is that the catalyst “fatigues,” where oxidation and the gradual accumulation of impurities reduces its efficiency over time. The catalyst must be replaced or reconditioned, and this is both an expensive and time-consuming process that incurs great expense to the user.

A third drawback of the F-T process is that a reasonable return on investment can only be achieved for very large F-T facilities. This precludes building a transportable or small system that can be taken to remote sources of gas. Approximately one-third of all natural gas is said to be “stranded”—that is, it is located at a place where it cannot be accessed at a reasonable cost. Finally, the F-T process is not species-specific. That is, it is not possible to tune the process to produce a single final long chain hydrocarbon. By its very nature, it produces a wide distribution of hydrocarbons, thereby requiring the subsequent separation of these products using fractionation, another large, expensive process.

### SUMMARY OF THE INVENTION

Broadly speaking, embodiments of the present invention provide excitation via monochromatic light at a particular wavelength. In one embodiment, a method for optical excitation of chemical species for enhanced chemical reaction is disclosed. The method includes providing a first chemical to a first illumination chamber and providing a second chemical to a second illumination chamber. The first chemical is illuminated with monochromatic light of a first predefined wavelength, wherein the first chemical is placed in an excitation state, and wherein the excitation state results in the molecules of the first chemical being more likely to react with other molecules. Similarly, the second chemical is illuminated with monochromatic light of a second predefined wavelength, wherein the second chemical is placed in an excitation state, and wherein the excitation state results in the molecules of the second chemical being more likely to react with other molecules. Next, the first chemical in the excitation state and the second chemical in the excitation state are provided to a reaction chamber, wherein molecules of the first chemical bond with molecules from the second chemical in a predefined manner to form a final product. Optionally, the unreacted molecules of the first chemical can be provided back to the first illumination chamber, and the unreacted molecules of the second chemical can be provided back to the second illumination chamber for further processing. That is, additional illumination and reaction of the unreacted first chemical and second chemical can be performed to form additional final product. Once produced the final product can be provided to a local storage. Optionally, the first and/or second chemical can be compressed prior to illumination.

A further method for optical excitation of chemical species for enhanced chemical reaction is disclosed in an additional embodiment. The method includes providing methane to an illumination chamber and illuminating the methane with monochromatic light of a predefined wavelength. This results in the methane being placed in an excitation state that results in the molecules of the methane bonding with other molecules to form propane. The methane in the excitation state can be provided to a reaction chamber. Further, the wavelength of the monochromatic light can be in the range of about 405 nm to 421 nm. Optionally the methane can be compressed prior to illumination. In addition, the unreacted molecules of the methane can be provided back to the illumination chamber for further processing.

A system for optical excitation of chemical species for enhanced chemical reaction is disclose in a further embodiment of the present invention. The system includes an illumination chamber and a monochromatic light having a predefined wavelength. The monochromatic light provides illumination to a chemical within the illumination chamber causing the molecules of the chemical to enter an excited state. The system further includes a reaction chamber in fluid communication with the illumination chamber. In operation, the molecules of the chemical in an excited state are provided to the reaction chamber and bond with other molecules to form a final product. For example, the chemical can be methane and the final product can be propane. In this case, the predefined wavelength can be 405 nm or, for example, 421 nm.

### BRIEF DESCRIPTION OF THE DRAWINGS

The invention, together with further advantages thereof, may best be understood by reference to the following description taken in conjunction with the accompanying drawings in which:

FIG. 1 illustrates a system for optical excitation of chemical species for enhanced chemical reaction, in accordance with an embodiment of the present invention;

FIG. 2 is a flowchart showing a method for optical excitation of a chemical species for enhanced chemical reaction, in accordance with an embodiment of the present invention;

FIG. 3 illustrates a system for optically exciting methane gas molecules into a longer chain hydrocarbon, such as propane, in accordance with an embodiment of the present invention; and

FIG. 4 is a flowchart showing a method 400 for optically exciting methane gas molecules into a longer chain hydrocarbon, such as propane, in accordance with an embodiment of the present invention.

### DETAILED DESCRIPTION OF THE EMBODIMENTS

Embodiments of the present invention relate to a system and method for enhanced chemical reactions via optical excitation of molecules. Excitation in the present disclosure refers to moving electrons from one orbital up to a higher level orbital. Embodiments of the present invention provide this excitation via monochromatic light at a particular wavelength.

Photons can be used as a means to get molecules moving, such as spinning or vibrating. Embodiments of the present invention use monochromatic light at a particular wavelength to find an excited state of a particular molecule that results in the molecule being more reactive for a reasonable amount of time. The molecules are then illuminated with the particular wavelength of monochromatic light to achieve enhanced reactions.

In this excited state, the molecules are more likely to react with other molecules. Random molecular motion of these molecules causes them to collide with other similarly excited molecules, and in some cases these collisions cause a chemical bonding. The specific wavelength of light that excites a molecule to such a state is based on the properties and atomic structure of the particular molecule, and can be different for different chemical reactions. It is possible that there might be intermediate states between the original molecule and the final reaction product. Such intermediate states may or may not be important to the overall process of optical conversion without detracting from the present invention. As such, embodiments of the present invention provide a system and method for enabling the rapid reaction of a chemical species with itself or other chemical species using carefully chosen wavelengths of light to enhance the reaction rate.

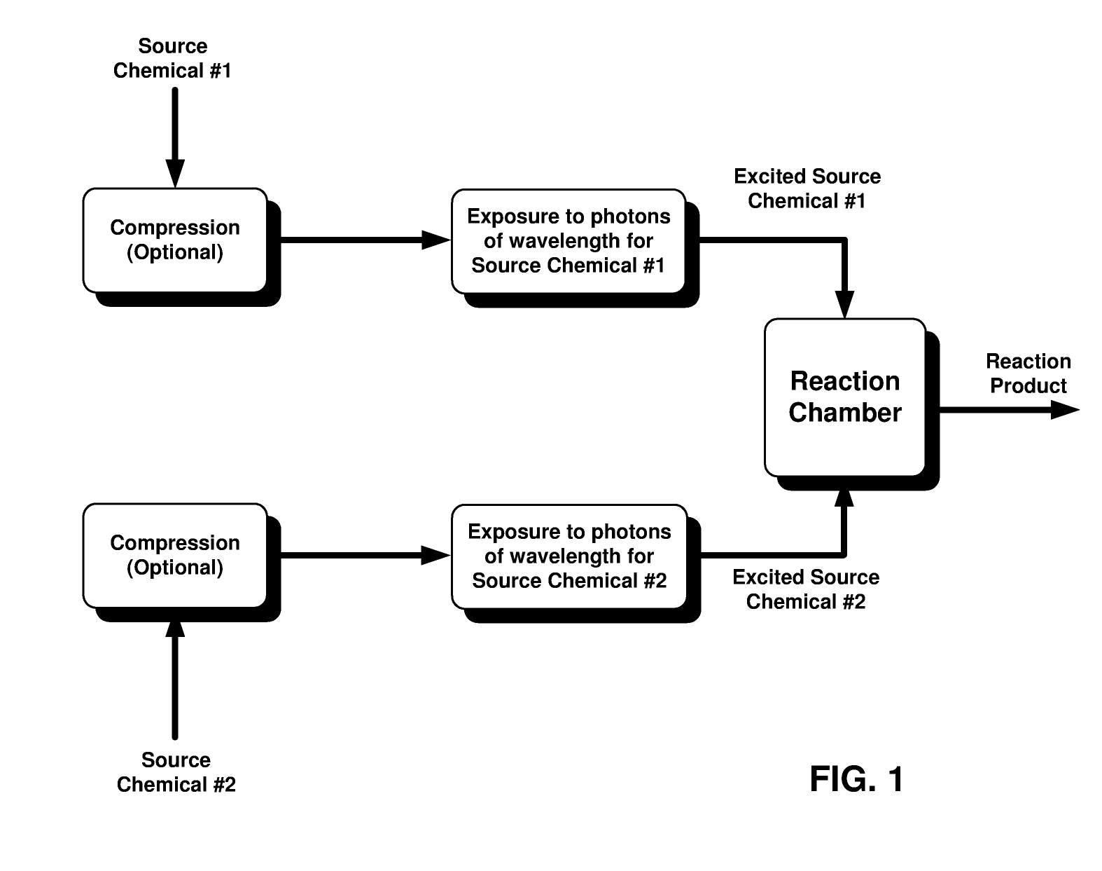

For example, FIG. 1 illustrates a system for optical excitation of chemical species for enhanced chemical reaction, in accordance with an embodiment of the present invention. As illustrated in FIG. 1, two source chemicals that are to be combined are provided to the system. These chemicals can be in any form, such as a gas or liquid, and can be different from one another. Optionally, one or both chemicals can be compressed for enhanced illumination and reaction, depending on the exact chemicals utilized and the needs of the particular system.

After the optional compression, the chemicals are provided to an illumination chamber where each chemical is illuminated by its own private excitation wavelength that results in the desired excitation state. For example, as will be described in greater detail subsequently, methane gas can be illuminated by monochromatic light at a wavelength of 421 nanometers (nm) to cause the methane gas to react to form longer chain hydrocarbons. In general, the desired excitation state results in the molecules of the particular chemical to bond in a desired manner with the molecules of the other chemical provided to the system.

For example, in the example of FIG. 1, source chemical 1 is illuminated by light, generally monochromatic light at a particular wavelength 1. Simultaneously, source chemical 2 is illuminated by monochromatic light of wavelength 2. In one embodiment, wavelength 1 selectively excites chemical 1, while wavelength 2 selectively excites chemical 2. The excited species of chemical 1 and chemical 2 then are allowed to mix with one another, such that the molecules of source chemical 1 bond with the molecules of source chemical 2 in a desired manner. This reaction chamber may be coincident with the two excitation chambers, or may be a separate, subsequent chamber, depending on the lifetimes of the excited states and the flow rate of the species. Upon colliding, some fraction of the excited source chemicals is provided to a reaction chamber where the molecules of the excited source chemicals bond in the desired manner to form the reaction product.

As will be appreciated by those skilled in the art after a careful reading of the present disclosure, embodiments of the present invention are highly efficient, use low energy consuming solid-state lasers, obviate the need for physical catalysts, and can operate on a small scale that is potentially transportable to remote locations. For example, embodiments of the present invention can be implemented as modules that are subsequently chained in sequence to create a system that converts a source gas (such as natural gas) to a target longer chain hydrocarbon (such as butane, gasoline, or diesel fuel). The present invention describes a system that achieves this goal through the use of carefully selected light sources (ideally, low cost, compact solid state lasers) and a supporting excitation/reaction system that allows the extraction of a final reaction product from the system right on site. Moreover, embodiments of the present invention can be applied to reactants in a liquid state, as well as a gas state.

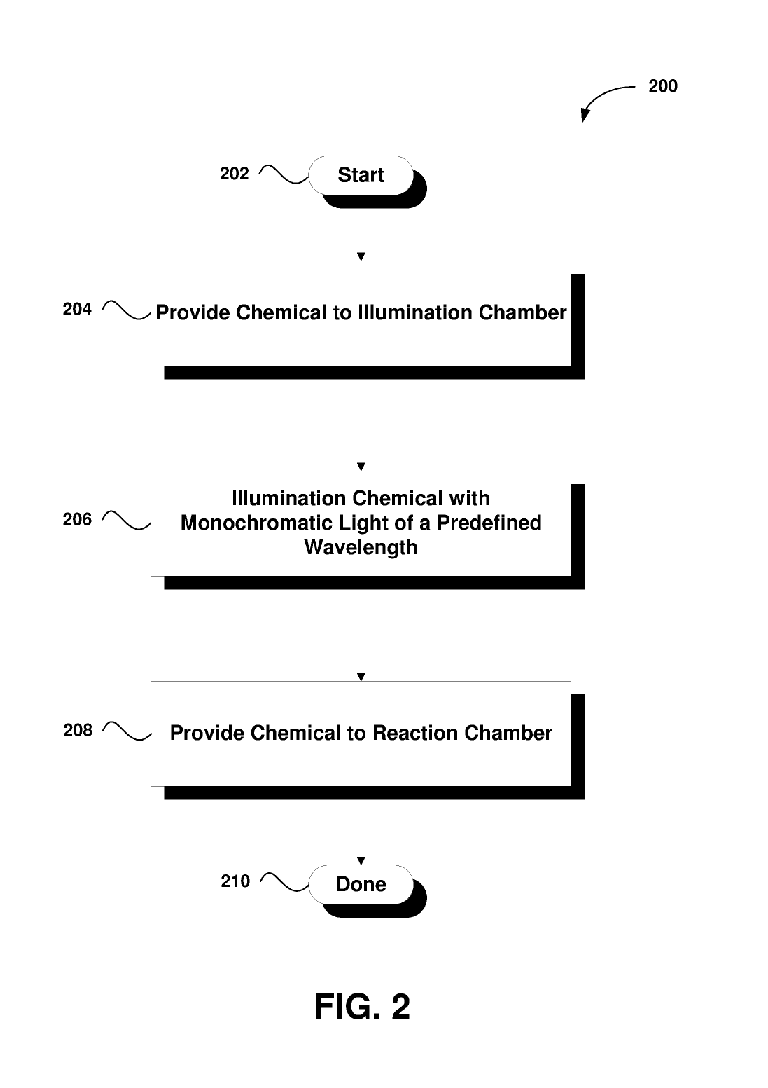

FIG. 2 is a flowchart showing a method 200 for optical excitation of a chemical species for enhanced chemical reaction, in accordance with an embodiment of the present invention. In an initial operation 202, preprocess operations are performed. Preprocess operations can include, for example, selecting a wavelength for the monochromatic light in the illumination chamber, selecting a chemical to excite, and further preprocess operations that will be apparent to those skilled in the art with hindsight provided after a careful examination of the present disclosure.

In operation 204, a chemical is provided to an illumination chamber. The chemical can be in any form, such as a gas or liquid, and can be optionally compressed for enhanced illumination and reaction, depending on the exact chemical utilized and the needs of the particular system.

The chemical then is illuminated with a monochromatic light of a predefined wavelength, in operation 206. As noted above, the predefined wavelength is selected such that illumination by the selected wavelength results in the desired excitation state of the chemical molecules. For example, noted previously, methane gas can be illuminated by monochromatic light at a wavelength of 421 nm to cause the methane gas to react to form longer chain hydrocarbons. In general, the desired excitation state results in the molecules of the particular chemical to bond in a desired manner with the molecules of the same or another chemical provided to the system.

The chemical in the excitation state is then provided to a reaction chamber where the molecules of the chemical bond with other molecules in a predefined manner. The reaction chamber can be coincident with the illumination chamber, or can be a separate, subsequent chamber, depending on the lifetimes of the excited states and the flow rate of the species. The molecules of the excited source chemical bond in the desired manner to form the reaction product.

Post process operations then are performed in operation 210. Post process operations can include, for example, returning an unreacted portion of the chemical back to the illumination chamber for further processing, storing the reacted portion of the chemical (i.e., the portion wherein the molecules bonded in the desired manner) to a local storage, and other post process operations that will be apparent to those skilled in the art with the hindsight afforded after a careful reading of the present disclosure. In addition to combining multiple chemicals, embodiments of the present invention can be utilized for unification of a single chemical to produce longer molecular chains that are more attractive than the original source molecule.

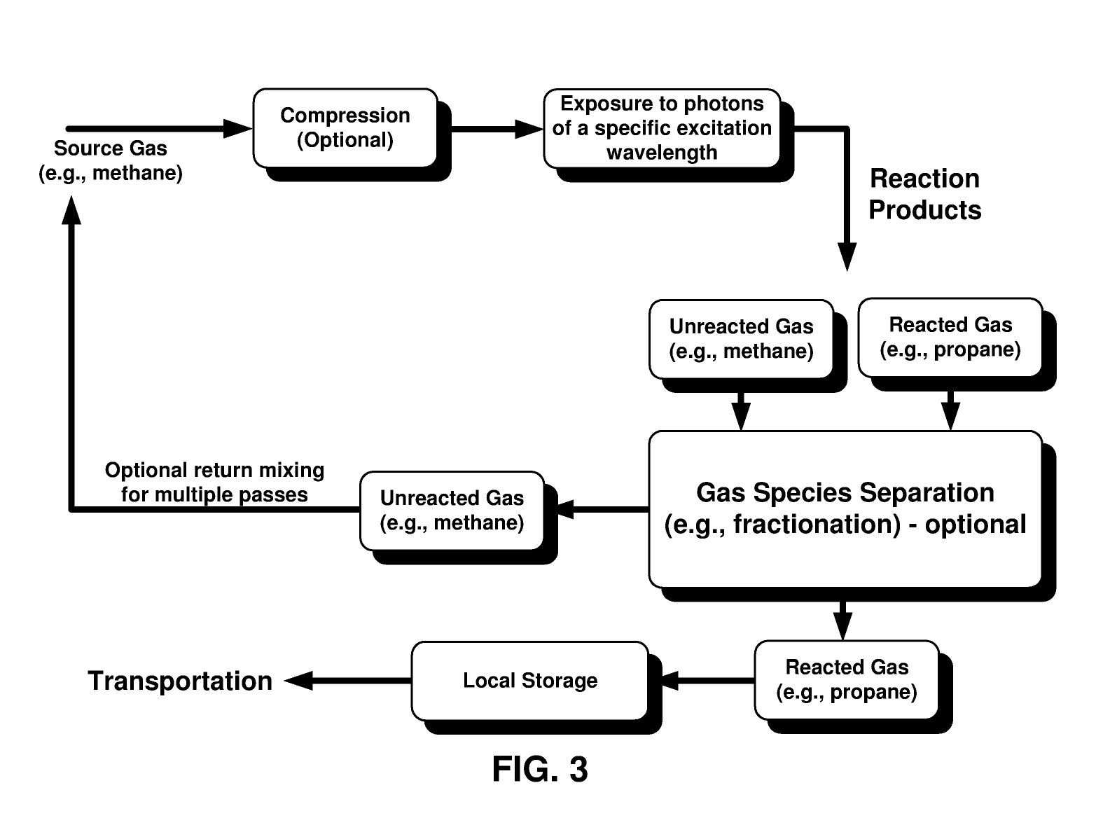

FIG. 3 illustrates a system for optically exciting methane gas molecules into a longer chain hydrocarbon, such as propane, in accordance with an embodiment of the present invention. It has been observed in the laboratory that methane molecules exhibit an excited state with a measurable lifetime when excited with monochromatic light at a wavelength of 421 nanometers (nm). It has been further observed that the excitation of methane molecules by photons of this wavelength is highly efficient, with efficiencies (number of excited molecules/number of photons) in excess of 50%. It has also been observed in the laboratory that such excited methane molecules react to form propane molecules.

Solid-state lasers present an attractive, compact option for creating a dense, high intensity illumination system for optical conversion of methane to propane. Unfortunately, lasers of the desired wavelength (421 nm) are not presently commercially available. However, a very inexpensive solid-state laser with a very close wavelength (405 nm) DOES exist and is used extensively in the commercial marketplace. The 405 nm lasers have a light emission spectrum that peaks at 405 nm, but all of the photons have slightly more energy than those of an ideal 421 nm laser. Since 405 nm photons have less than 3% MORE energy than the ideal laser, it was found that the low cost 405 nm lasers can be used to excite methane molecules. It was found that the small difference in energy off the peak absorption wavelength of 421 nm did not appreciably reduce the reaction rate of methane molecules to propane molecules. In fact, the net benefit of the commercially available laser more than compensated for any reduction in reaction efficiency due to wavelength detuning.

Therefore, in light of these observations, one embodiment of the present invention employs photons of wavelength 405 nm to optically excite methane molecules to a long lived excited state with a reduced activation potential. This causes the excited methane to react with and bond to other excited methane molecules, producing (in the observed embodiment) propane molecules.

There are many possible configurations for causing an input stream of gas (in the specific instance of FIG. 3, the input gas is methane) to be exposed to excitation light of a specific wavelength, and then separating the results into streams of the desired end product (such as propane) and unreacted gas. The end result can be sent on to a storage tank of some sort, and the unreacted gas can optionally be returned back to mix with the original input stream for an additional pass. This disclosure also presents a configuration in which a raw input stream is stored in a collection tank and later sent into an exposure station where the molecules are exposed to a specific wavelength of light. The result is a mixture of the desired end product and the source gas. These can be separated through standard means known to those skilled in the art, and the unreacted gas can then be returned to mix with the input stream for a second (or even third, fourth, etc.) pass.

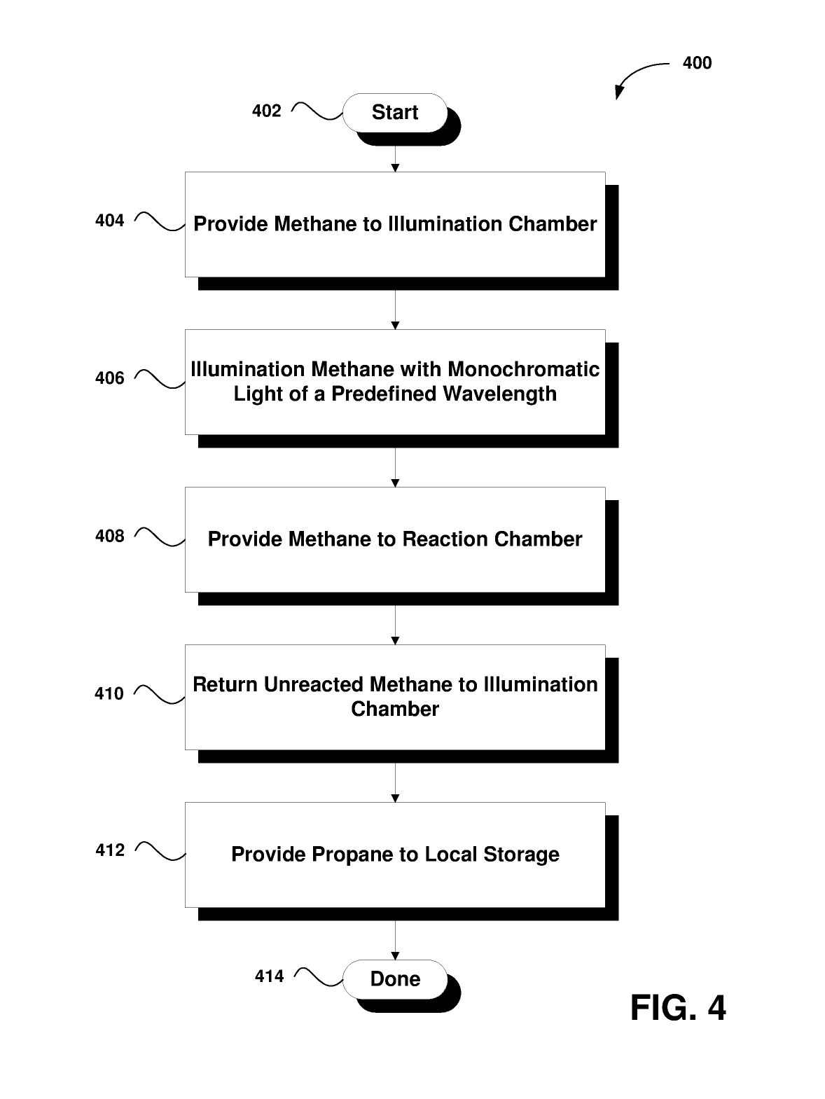

FIG. 4 is a flowchart showing a method 400 for optically exciting methane gas molecules into a longer chain hydrocarbon, such as propane, in accordance with an embodiment of the present invention. In an initial operation 402, preprocess operations are performed. Preprocess operations can include, for example, selecting a wavelength for the monochromatic light in the illumination chamber (typically in the range of about 405 nm and 421 nm), determining whether to use compression prior to illumination, and further preprocess operations that will be apparent to those skilled in the art with hindsight provided after a careful examination of the present disclosure.

In operation 404, methane is provided to an illumination chamber. As above, the methane can be in any form, such as a gas or liquid, and can be optionally compressed for enhanced illumination and reaction, depending on the needs of the particular system.

The methane then is illuminated with a monochromatic light of a predefined wavelength, in operation 406. As noted above, the predefined wavelength is selected such that illumination by the selected wavelength results in the desired excitation state of the chemical molecules. As discussed previously, it has been observed in the laboratory that methane molecules exhibit an excited state with a measurable lifetime when excited with monochromatic light at a wavelength of 421 nm. It has been further observed that the excitation of methane molecules by photons of this wavelength is highly efficient, with efficiencies (number of excited molecules/number of photons) in excess of 50%. It has also been observed in the laboratory that such excited methane molecules react to form propane molecules. However, monochromatic light at wavelength 405 nm can be used excite methane molecules. The small difference in energy off the peak absorption wavelength of 421 nm does not appreciably reduce the reaction rate of methane molecules to propane molecules.

In operation 408, the methane in the excitation state is then provided to a reaction chamber where the molecules bond with other molecules in a predefined manner. As noted above, the excited methane molecules react with and bond to other excited methane molecules, producing propane molecules.

In optional operation 410, the unreacted methane molecules are returned to the illumination chamber for further processing. In some embodiments some methane molecules will fail to react with other methane molecules. As a result, the two chemicals can remain in the reaction chamber after chemical reaction: 1) methane formed from unreacted methane molecules, and 2) propane formed from reacted methane molecules that have bonded to form propane molecules. Hence, in optional operation 410, the unreacted methane molecules can be optionally returned to the illumination chamber for further processing, depending on the needs of the particular system.

In operation 412, the propane molecules are provided to local storage. The end result can be sent on to a storage tank of some sort, and the unreacted gas can optionally be returned back to mix with the original input stream for an additional pass as noted in operation 410. In one embodiment the raw input stream is stored in a collection tank and later sent into an exposure station where the molecules are exposed to a specific wavelength of light. The result is a mixture of the desired end product and the source gas. These can be separated through standard means known to those skilled in the art, and the unreacted gas can then be returned to mix with the input stream for a second (or even third, fourth, etc.) pass.

Although the foregoing invention has been described in some detail for purposes of clarity of understanding, it will be apparent that certain changes and modifications may be practiced within the scope of the appended claims. Accordingly, the present embodiments are to be considered as illustrative and not restrictive, and the invention is not to be limited to the details given herein, but may be modified within the scope and equivalents of the appended claims.

What is claimed is:

1. A method for optical excitation of chemical species for enhanced chemical reaction, comprising:
providing a first chemical to a first illumination chamber;
providing a second chemical to a second illumination chamber;
illuminating the first chemical with monochromatic light of a first predefined wavelength, wherein the first chemical is placed in an excitation state, and wherein the excitation state results in the molecules of the first chemical being more likely to react with other molecules;
illuminating the second chemical with monochromatic light of a second predefined wavelength, wherein the second chemical is placed in an excitation state, and wherein the excitation state results in the molecules of the second chemical being more likely to react with other molecules; and
providing the first chemical in the excitation state and the second chemical in the excitation state to a reaction chamber, wherein molecules of the first chemical bond with molecules from the second chemical in a predefined manner to form a final product.

2. The method recited in claim 1, further comprising providing unreacted molecules of the first chemical back to the first illumination chamber.

3. The method recited in claim 2, further comprising providing unreacted molecules of the second chemical back to the second illumination chamber.

4. The method recited in claim 3, further comprising additional illumination and reaction of the unreacted first chemical and second chemical to form additional final product.

5. The method recited in claim 1, further comprising providing the final product to a local storage.

6. The method recited in claim 1, further comprising compressing the first chemical prior to illumination.

7. The method recited in claim 1, further comprising compressing the second chemical prior to illumination.

8. A method for optical excitation of chemical species for enhanced chemical reaction, comprising:
providing methane to an illumination chamber;
illuminating the methane with monochromatic light of a predefined wavelength, wherein the methane is placed in an excitation state, and wherein the excitation state results in the molecules of the methane bonding with other molecules to form propane.

9. The method as recited in claim 8, further comprising providing the methane in the excitation state to a reaction chamber.

10. The method as recited in claim 8, wherein the wavelength is in the range of about 405 nm to 421 nm.

11. The method of claim 8, further comprising compressing the methane prior to illumination.

12. The method as recited in claim 8, further comprising providing unreacted molecules of the methane back to the illumination chamber for further processing.

13. A system for optical excitation of chemical species for enhanced chemical reaction, comprising:
an illumination chamber;
a monochromatic light having a predefined wavelength, wherein the monochromatic light provides illumination to a chemical within the illumination chamber causing the molecules of the chemical to enter an excited state; and
a reaction chamber in fluid communication with the illumination chamber, wherein molecules of the chemical in an excited state are provided to the reaction chamber and bond with other molecules to form a final product.

14. The system as recited in claim 13, wherein the chemical is methane.

15. The system as recited in claim 14, wherein the final product is propane.

16. The system as recited in claim 15, wherein the predefined wavelength is 405 nm.

17. The system as recited in claim 15, wherein the predefined wavelength is 421 nm.
